(19) United States
(12) Patent Application Publication
(10) Pub. No.: US 2007/0111297 A1
(43) Pub. Date: May 17, 2007

(54) Portable micro-flow managing system using infinitesimal pressure control

(75) Inventors: Sang-Joon Lee, Pohang-city (KR); Guk-Bae Kim, Pohang-city (KR)

Correspondence Address: MARSHALL, GERSTEIN & BORUN LLP, 233 S. WACKER DRIVE, SUITE 6300, SEARS TOWER, CHICAGO, IL, 60606, US

(73) Assignee: POSTECH ACADEMY-INDUSTRY FOUNDATION, Pohang-city (KR)

(21) Appl. No.: 11/595,116

(22) Filed: Nov. 9, 2006

(30) Foreign Application Priority Data
Nov. 11, 2005 (KR) 10-2005-0107973

(51) Int. Cl. C12M 1/36; C12M 1/34

(52) U.S. Cl. 435/286.6; 435/287.2; 435/288.5

(57) ABSTRACT

A micro-flow managing system is provided. The micro-flow managing system includes a pressure generator having a plurality of pressure chambers and a plurality of depressurizing chambers which generate respective set pressures; a valve kit having a plurality of selection valves, and a valve controller which control the selection valves to select the set pressure transferred to the biochip. Each of the selection valves is configured such that one of the set pressures corresponding to each of the pressure chambers is transferred to a biochip through a coupling path.

### BACKGROUND OF THE INVENTION

(a) Field of the Invention

The present invention relates to a portable micro-flow managing system using infinitesimal pressure control method, and more particularly, to a biochip system including lab-on-a-chip and a micro-flow managing system available for fluidic devices and industry fields requiring micro-flow managing.

(b) Description of the Related Art

In the past time, in most cases, one technology was dedicated to one product. However, recently, technologies in various fields are cooperated and integrated to develop and manufacture a new product in a short time.

In the early twenty-first century, the biotechnology(BT) can accomplish a rapid progress such as a completion of human genome map, because information technology(IT) can be applied to the data processing and analysis.

Such a fusion of several technologies is found as a general tendency in the recent research and development. The most active and attractive field is the fusion of the IT representing the twentieth century and the BT representing twenty-first century.

In addition, due to the advances of nano technology(NT), a new product that has not been expected before can be made.

A biochip, that is, a small-scale device to test bionic material such as DNA or protein in a chip is one of the representative fusion technology formed by using BT-IT-NT technologies.

Conventional bio chips have been used for simply analyzing the products in the biotechnology field. However, recently, the bio chip is employed widely to various industry applications.

The biochip is a product to test bio-molecules such as DNA and protein on a small substrate made of such as glass, silicon, and polymer.

For example, a biochip integrated with DNA is called a DNA chip, and a biochip integrated with protein is called a protein chip.

The biochips are classified into a microarray chip and a micro fluidics chip.

The microarray chip is a biochip formed by arranging and attaching thousands, or tens of thousands of DNA or protein with a predetermined interval and mounting a to-be-analyzed material to analyze its reaction. The aforementioned DNA chip and protein chip are representative ones of the microarray chip.

The micro fluidics chip is a biochip for analyzing an aspect of reaction of a small quantity of a to-be-analyzed material which flows with various biomolecules integrated in the chip. This micro fluidics chip is also called as a lab-on-a-chip.

Such a micro fluidics chip is constructed by integrating functions of a pump, a valve, a reactor, an extractor, or a separator essential to sample preparation of an automatic analyzer for analyzing (bio)chemical materials and a sensor technology on the same chip.

Currently, some biochips for analyzing DNA are commercialized on the market. However, although the lab-on-a-chip or micro fluidics chip which requires precise control of micro-scale flow therein has many applications, a compact handheld system of lab-on-a-chip or micro fluidics chip has not been commercialized as a micro-Total Analysis System.

Such a biochip is expected to be widely used in public fields such as health and medical service. In near future, the biochip is also expected to be used as means for monitoring freshness or pollution of foods, drinks, home environments, and the like.

Therefore, the biochip will widely spread and contribute greatly to improve human life or medical environment.

Similarly to computers which drew a new paradigm in the IT industry, the biochip as bionics and environment diagnosis system will bring out a new paradigm in the BT industry and change our life style.

As the biochip related research works are active, the biochip is not limited to research of DNA or protein in laboratories of college or institute, but its application field is gradually expanded into general subjects such as testing of blood, saliva, and physiology secretion.

However, although the biochip itself is highly developed, the elemental technologies such as a precise flow control technology for effectively managing micro-scale flow inside the chip or a measuring technology for detecting test result is insufficiently developed.

In other words, the current biochip system has not been fully developed into a multi-functional, portable micro-Total Analysis System.

The conventional biochips have a simple structure, because DNA or protein which is previously separated, refined, and amplified in a lab is mounted on the chip directly. In order to make the lab-on-a-chip available even for the ordinary person, DNA and protein needs to be extracted from blood or somatic cell on the same chip in the beginning stage.

For this reason, micro channels need to be designed to pass a target DNA and washing solution on the chip. The micro channel can be used as a path for transporting fluids in the processes of moving, mixing, reacting, separating, diluting, refining the sample and reacting reagents, and the like.

In many cases, in order to obtain optimal processes, the flow of reactants or bio samples may be controlled to open and close. In this case, the flow of fluids can be controlled with pumps or valves provided along the channels.

Conventionally, an injection pump or an electric actuator using a high voltage (a few kV) has been used to manage the fluids in the actual chip.

Therefore, peripherals used for such a flow managing system are too large and heavy to be applied to the portable biochip. In addition, the internal fluids flow in the biochip cannot be effectively controlled.

In the currently available commercialized biochips, flow can be controlled simply with a very simple actuating mechanism.

### SUMMARY OF THE INVENTION

Thus, the present invention provides a small, light, portable flow managing system for a biochip capable of effectively controlling micro-scale flow in the chip.

According to an aspect of the present invention, there is provided a micro-flow managing system comprising: a pressure generator having a plurality of pressure chambers which generate respective set pressures; a valve kit having a plurality of selection valves, each of the selection valves configured such that one of the set pressures corresponding to each of the pressure chambers is transferred to the biochip through a coupling path; and a valve controller which controls the selection valves to select the set pressure transferred to the biochip.

In the above aspect of the present invention, the pressure chambers may comprise a small pressurizing chamber, a small depressurizing chamber, or the combination thereof, without supplying a separate energy, and the valve controller may include a computer to control the selection valves according to input information given by an operator and preset input program.

The micro-flow managing system having such a construction is inexpensive, and the entire size of the control device is very small and light compared to the conventional flow control device. In addition, the micro-flow in the chip can be controlled effectively by using the valve kit capable of programming the control process.

Therefore, in commercializing a multipurpose portable biochip, the micro-flow managing system according to the aspect of the present invention can be an essential core technology.

The micro-flow managing system according to the aspect of the present invention can be very useful in developing the portable micro-Total Analysis System.

In addition, as functions of the biochip become more complicated, more accurate micro-flow managing techniques will be required. Therefore, the micro-flow managing system according to the aspect of the present invention will be more useful.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram showing a schematic diagram of micro-flow managing system according to an embodiment of the present invention;

FIG. 2 is a bottom view showing a valve kit shown in FIG. 1; and

FIG. 3 is an enlarged bottom view showing a selection valve shown in FIG. 2.

### DETAILED DESCRIPTION OF THE PREFERRED EMBODIMENTS

Hereinafter, exemplary embodiments of the present invention will be described in detail with reference to the attached drawings.

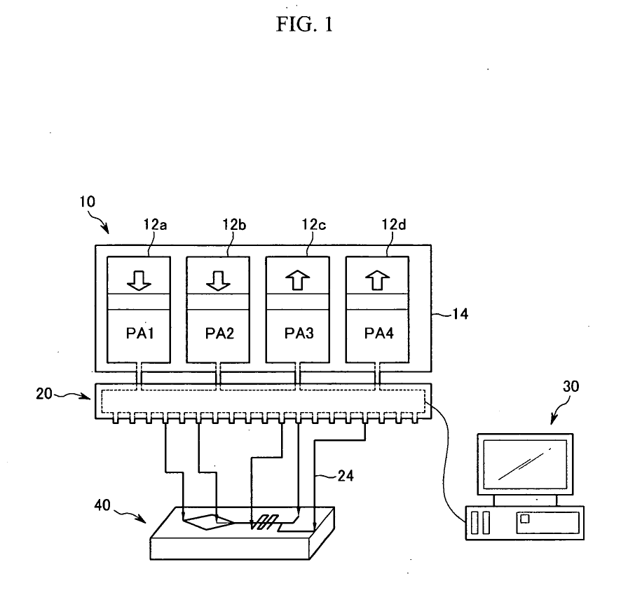

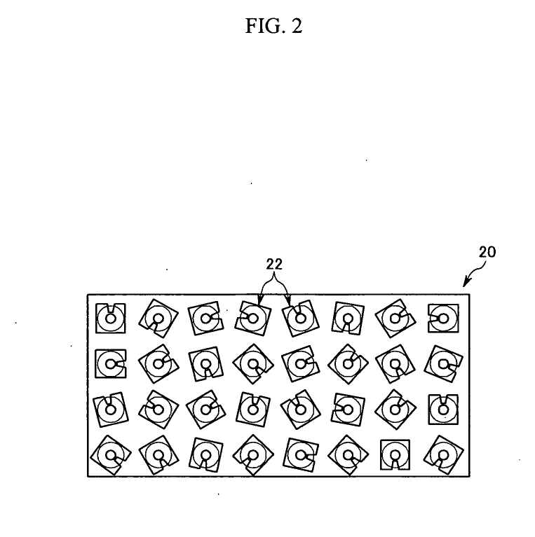

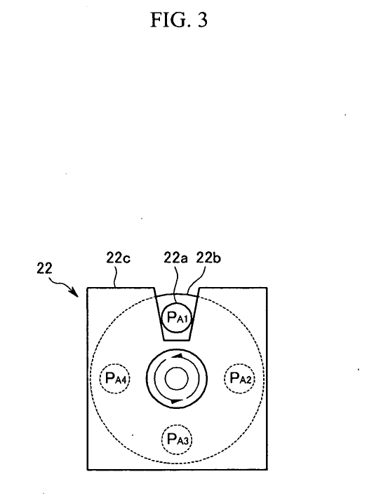

FIG. 1 is a schematic diagram showing a construction of the micro-flow managing system for the biochip according to the embodiment of the present invention, FIG. 2 is a bottom view showing a valve kit shown in FIG. 1, and FIG. 3 is an enlarged bottom view showing a selection valve shown in FIG. 2.

As shown in FIG. 1, the micro-flow managing system according to the embodiment of the present invention comprises a pressure generator 10, a valve kit 20, and a valve controller 30.

The pressure generator 10 comprises a plurality of pressure chambers 12a, 12b, 12c, and 12d, the pressure chambers 12a, 12b, 12c, and 12d are fixed to a chamber housing 14.

The pressure chambers comprises a pressurizing chamber 12a and 12b generating a positive (+) pressure, and a depressurizing chamber 12c and 12d generating a negative (−) pressure. Hence, the depressurizing chamber 12c and 12d may be used to extract a sample from a biochip 40 or to mix the sample.

The numbers and types of such pressure chambers 12a, 12b, 12c, and 12d may be changed variously, and the magnitude of the positive (+) pressure and the negative (−) pressure may also be set variously.

Although not shown in detail, the pressure chambers 12a, 12b, 12c, and 12d may comprise a cylinder, a piston body in the cylinder, and a pressure setting part setting the pressure inside the cylinder by operating the piston body.

In this case, the pressure setting part may be configured as a manual type or an automatic type. For the manual type, it may comprise a knob protruded outside the cylinder.

Because the manual type of pressure setting part may not require a separate power source for generating a pressure in the pressure chamber, the system size can be reduced. This reduction in size can contribute to the commercialization of the portable bio chip.

In addition, the automatic type of pressure setting part may comprise a separate actuator for operating the piston body according to an electric signal.

In addition, the pressure chambers 12a, 12b, 12c, and 12d may further comprise, a pressure sensing part, for example, a pressure sensor for sensing the pressure inside the cylinder.

In this case, an input signal sensed by the pressure sensor may be used as input data to the valve controller 30 to present output signal in video image or audio sound when the pressure reaches to the set pressure or if the pressure is lowered than the set pressure due to use of pressure.

The valve kit 20 coupled to the outlet of the pressure chambers 12a, 12b, 12c, and 12d comprises a plurality of selection valves 22.

Each of the selection valves 22 is configured such that one of the set pressures corresponding to the pressure chambers 12a, 12b, 12c, and 12d is transferred to the biochip through a coupling path 24, and may be configured as various forms.

For example, the selection valve 22 may comprise a valve body 22b having a plurality of channels 22a, a rotary valve 22c for operating only one channel selected from the channels 22a, and an actuator for opening the rotary valve 22c with a preset amount according to a signal from the valve controller 30.

The plurality of channels 22a is coupled to the pressure chambers 12a, 12b, 12c, and 12d respectively. Therefore, a pressure of one pressure chamber is transferred to the biochip 40 through only are channel 22a selected by the rotary valve 22c. Such action is made independently in each of the selection valves 12a, 12b, 12c, and 12d.

In other words, each of the selection valves 22 is connected to each of the inlet of the biochip 40 to control the flow in the inlets independently.

The selection valve 22 may be operated very fast within a few milliseconds (10⁻³ s) by manipulating on/off the rotary valve 22c by means used for the program of the valve controller 30.

Therefore, very small micro-scale flow can be implemented.

In addition, although not shown in FIG. 1, in case of installing two or more valves in one valve line to control the valves sequentially in a very short time, much small flow rate can be controlled.

In addition, the valve controller 30 may be implemented by a computer to control an actuator according to information input by an operator or preset input program. In this case, the computer may pressure information of the pressure chamber as a video image or an audio sound, depending on pressure signal detected by a pressure sensor.

In addition, the program may be programmed variously according to the type of the biochip.

According to such a construction, the selection valves are coupled to the pressure chambers through the microchannels provided to the selection valve, and the pressure outlets of the selection valves are coupled to the corresponding points of the biochip 40 by using the coupling path 24, so that the rotary valve in each selection valve 22 can be controlled individually by using the valve controller 30.

Therefore, the pressure applied to the corresponding point of the biochip 40 can be controlled effectively.

Accordingly, the flow of micro-scale fluids in the biochip can be controlled precisely.

For example, in case of the micro channel with the width of 0.1 mm and the height of 0.05 mm, the micro-flow managing system according to the embodiment of the present invention can control the flow in the microchannel within an accuracy of 0.01 mm.

Although the preferred embodiments of the present invention have been described, the present invention is not limited to the embodiments but may be modified in various forms without departing from the scope of the appended claims, the detailed description, and the accompanying drawings of the present invention.

For example, the micro-flow managing system of according the embodiment of the present invention may also be used as a flow managing system for medical instruments, and it is also within the scope of the present invention.

As described above, since the flow managing system for the biochip of the present invention controls the micro-scale flow in the biochip, the micro-scale flow in the biochip with complicated reaction stages can be controlled effectively.

For example, in case of the micro channel with the width of 0.1 m and the height of 0.05 mm, the micro-scale flow in the channel can be controlled within an accuracy of 0.01 mm, which corresponds to 50 nano-liters.

In addition, the size of the total system can be implemented to be very small compared to the conventional system. Therefore, a portable biochip can be easily fabricated for commercialization.

In addition, the flow managing system according to the present invention can be effectively used for various applications where several flow managing operations need to be performed out simultaneously on a multi-functional biochip.

What is claimed is:

1. A portable micro-flow managing system using infinitesimal pressure control, comprising:
a pressure generator having a plurality of pressure chambers which generate respective set pressures;
a valve kit having a plurality of selection valves, each of the selection valves configured such that one of the set pressures corresponding to each of the pressure chambers is transferred to a biochip through a coupling path; and
a valve controller which controls the selection valves to select the set pressure transferred to the biochip.

2. The portable micro-flow managing system of claim 1, wherein the valve controller includes a computer to control the selection valves according to information input by an operator and preset input program.

3. The portable micro-flow managing system of claim 2, wherein the selection valve comprises a valve body having a plurality of channels and a rotary valve for rotating to open only one channel selected from the channels.

4. The portable micro-flow managing system of claim 1, wherein the pressure chambers comprise a pressurizing chamber, a depressurizing chamber, or the combination thereof.

5. The portable micro-flow managing system of claim 4, wherein the valve controller includes the computer to control the selection valves according to information input by an operator and previously input program.

6. The portable micro-flow managing system of claim 5, wherein the selection valve comprises the valve body including the channels and the rotary valve for rotating to open only one channel selected from the channels.
